(19) United States
(12) Patent Application Publication
(10) Pub. No.: US 2004/0092958 A1
(43) Pub. Date: May 13, 2004

(54) Stereotactic wands, endoscopes and methods using such wands and endoscopes

(76) Inventors: Farhad M. Limonadi, Portland, OR (US); Edmund Frank, Portland, OR (US)

Correspondence Address: KLARQUIST SPARKMAN, LLP, 121 SW SALMON STREET, SUITE 1600, PORTLAND, OR, 97204, US

(21) Appl. No.: 10/416,913

(22) PCT Filed: Nov. 15, 2001
(86) PCT No.: PCT/US01/43863

(51) Int. Cl. A61B 19/00

(52) U.S. Cl. 606/129

(57) ABSTRACT

A stereotactic surgical device includes at least two light guides adapted to converge light to a predetermined extent at a predetermined distance from a predetermined location on the surgical device. The surgical device may further include a light detector and a processor to receive image information from the light detector. Logic of the surgical device is applied to the processor to cause the processor to determine when the light has converged to the predetermined extent and to provide a signal thereupon.

This application claims the benefit of U.S. Provisional Patent Application No. 60/249,780, filed Nov. 17, 2000.

### FIELD

The present invention relates to stereotactic surgical devices, and more particularly to stereotactic endoscopes and wands.

### BACKGROUND

Stereotactic surgical equipment permits precise, minimally invasive surgical procedures such as tumor removal, specimen extraction, photocoagulation, and other procedures. An endoscope generally includes an optical system such as series of lenses or a fiber bundle configured to relay an image from an observation point within the body of a patient to an exterior location for direct viewing by a physician, or for display on a video monitor. Various surgical tools can be associated with an endoscope such as, for example, tissue resectors and aspirators. These tools can be precisely placed and manipulated based on images transmitted by the endoscope.

The optical system of the endoscope may comprise a focal point, so that the observation point within the body comes into focus when a distal end of the endoscope is a predetermined distance from the observation point. Reliable positioning of an endoscope with respect to observation point is complicated by the “subjectivity” of the endoscopic focal point. In other words, an observation point that appears in focus to one observer may not appear in focus to a second observer, due to differences in the vision of the observers.

Stereotactic wands are similar in some respects to stereotactic endoscopes. However, wands are typically employed to establish the location of tissue features in the body with respect to an external frame of reference. Unlike endoscopes, wands typically do not include an optical system for relaying images of the body's interior back to an observer. For this reason, an observer has less visual information at their disposal for positioning the wand. Often, the wand is inserted until it makes contact with the tissue feature, distorting the tissue, and then the wand is pulled back to some extent. Deformations and distortions of the tissue feature resulting from contact with the wand may complicate reliable positioning of the wand with respect to the tissue feature, and increase risks associated with wand use, particularly in sensitive neurological or ophthamalogical procedures.

### SUMMARY

In one aspect, a stereotactic endoscope or stereotactic wand includes at least one lens to converge light to a predetermined extent at a predetermined distance from a predetermined location on the endoscope or wand. Alternatively, a light guide may be formed to converge the light without use of a converging lens or prism.

In another aspect, a light-assisted stereotactic body includes an opening to receive a non-light assisted surgical device, such as an endoscope, wand, or cannula. The body further includes at least one lens to converge light to a predetermined extent at a predetermined distance from a predetermined location on one of the device and the endoscopic body. Alternatively, a light guide may be formed to converge the light without use of a converging lens or prism.

In another aspect, a stereotactic system includes an imaging system configured to form an image of a tissue feature located at a predetermined distance from a location on an endoscope or wand. A focus system provides one or more light fluxes that converge at the predetermined distance.

A stereotactic surgical device may include a light detector and a processor to receive image information from the light detector, and may also include logic which, when applied to the processor, causes the processor to determine when the light has converged and to provide a signal thereupon to effect registration of the device.

### DRAWINGS

FIG. 1 is an illustration of an embodiment of a stereotactic endoscopic system.

FIGS. 2-4 illustrate a light-assisted stereotactic device embodiment in various aspects relative to a tissue feature.

FIG. 5 illustrates an embodiment of a light-assisted stereotactic device.

FIG. 6 illustrates an embodiment of a stereotactic system using an embodiment of a light-assisted stereotactic device.

FIG. 7 illustrates internal components of an embodiment of a light-assisted stereotactic device.

FIG. 8 illustrates an embodiment of a light-assisted stereotactic body which may be fitted to a conventional surgical device.

### DESCRIPTION

In the following description, references to “one embodiment” and “an embodiment” do not necessarily refer to the same embodiment, although they may.

In one embodiment, a stereotactic endoscope or wand comprises at least one lens to converge light to a predetermined extent at a predetermined distance from a predetermined location on the endoscope or wand. In one embodiment, the predetermined extent of convergence is substantially a point, and the predetermined location on the endoscope or wand is a location on a distal end of the device. In another embodiment, the endoscope or wand comprises a light guide formed to converge the light without use of a converging lens or prism.

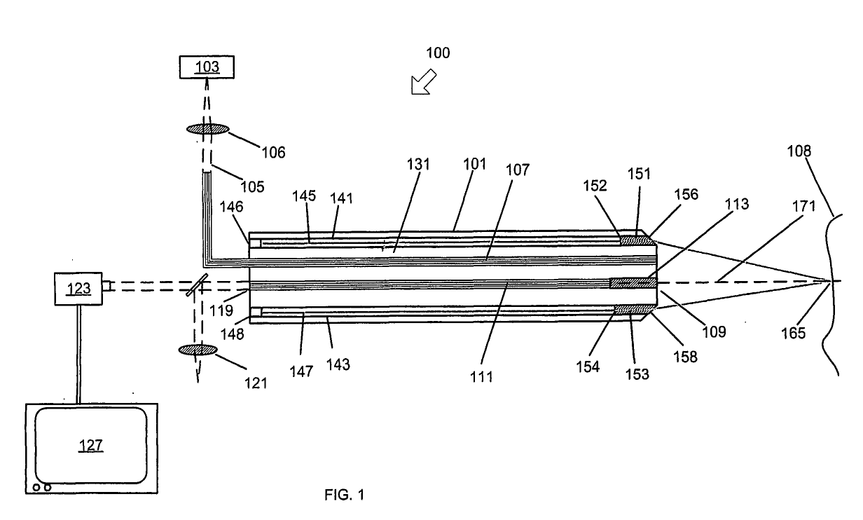

With reference to FIG. 1, an endoscopic system 100 includes an endoscope embodiment 101. The system 100 comprises a light source 103 that directs a light flux 105 into a condenser lens 106. The condenser lens 105 collimates the light flux into an optical fiber bundle 107. The light flux 105 exits a distal end 109 of the endoscope 101 and is incident to a tissue feature 108. The light flux 105 may be monochromatic or polychromatic. Exemplary light sources 103 include a laser diode or other laser source, a light emitting diode (LED) or combination of LEDs, a quartz-halogen lamp or other incandescent light source, and a fluorescent light source. To reduce the effects of light exposure on the tissue feature 108, the light flux 105 may be filtered to remove wavelengths such as infrared and ultraviolet. Fiber bundles are of course only one manner of propagating a light flux, and other manners, such as lens tubes, may also be employed.

The endoscope 101 further comprises a fiber bundle 111 and an objective lens 113. The fiber bundle 111 may be coherent, e.g. the ends of the fibers of the bundle may be similarly arranged on both ends of the bundle. In alternative embodiments a series of lens elements or lenses may be used in place of the fiber bundle 111, or a gradient index rod may be used. If the endoscope 101 includes a gradient index rod, a separate objective lens 113 may be omitted. When used, the objective lens 113 may be a gradient index lens, biconvex lens, planoconvex lens, or other lens element. The objective lens 113 may also be an achromatic lens or other lens that includes multiple lens elements. The objective lens 113 forms an image of the tissue feature 108. The image is relayed by the fiber bundle 111 to a proximal end 119. An eyepiece lens 121 may be provided to form an image of the tissue feature 108 for direct viewing by an operator of the system 100. Alternatively, a camera 123 may be provided to receive an image of the tissue feature 108 and relay the image to a video monitor 127 for display.

The endoscope 101 may also include a cannula 131 to provide access to a region of the tissue feature 108. The cannula 131 may be employed, for example, to channel fluids and/or gasses to and from the region of the tissue feature 108. The cannula 131 may provide access the region for surgical tools and/or intense light fluxes. The cannula 131 may also be useful in channeling fluids to flush debris and occlusions from the distal end 109 of the endoscope 101. Some embodiments may comprise more than one cannula 131. Of course, the cannula 131 may be omitted from some embodiments of the endoscope 101 (for example, where the endoscope is intended primarily for observation of the tissue feature 108). The diameter of the cannula 131 may, in some embodiments, be substantially larger than the diameter of the objective lens 113.

In one embodiment, light guides 141, 143 may be provided for the transmission of light beams 145, 147 from the light beam sources 146, 148 to the lenses 151, 153, respectively. In one embodiment, the lenses 151, 153 are gradient index lenses. The light beam sources 146, 148 may include laser diodes and laser diode beam shaping optics so that the beams 145, 147 are substantially collimated with respect to an overall length L of the endoscope 101. In one embodiment, a substantially collimated beam is one having an angular divergence such that a diameter of the beam does not vary by more than about a ratio of 2:1 along L. In one embodiment, laser diodes that emit radiation at wavelengths of between about 600 nm and 680 nm may be employed as the light beam sources 146, 148. Of course, laser diodes which produce other wavelengths may also be used. In alternative embodiments, the light beam sources 146, 148 may be omitted, and a light flux from one or more lasers such as laser diodes or gas lasers (e.g., helium-neon lasers) can be directed to the endoscope 101 through an optical fiber or fibers. A laser beam from a single source can be split as an alternative to providing two sources for the beams 145, 147.

The lenses 151, 153 include respective entrance surfaces 152, 154 and exit surfaces 156, 158, the exit surfaces 156, 158 angled with respect to the entrance surfaces 152, 154. The lenses 151, 153 may thus operate as prisms to direct the beams 145, 147 toward a point 165 The lenses 151, 153 may also focus the beams at the point 165. The point 165 may be selected to be a predetermined distance from the distal end 109 of the endoscope 101. The point 165 may be located a predetermined distance from an reference location on the endoscope 101, not just from the distal end 109.

In an alternate embodiment, the light guides 141, 143 maybe formed to direct the beams 145, 147 toward the point 165. For example, the light guides 141, 143 may comprise optical fibers which are angled at the distal end 109 of the endoscope 100 to converge the beams 145, 147 toward the point 165. In this case, the lenses 151, 153 may be omitted or may be non-converging lenses used to terminate the light guides 141, 143.

The size of the spot produced by the beams 145, 147 at the point 165 may be determined, at least in part, by the focal lengths of the lenses 151, 153 and the diameters of the beams 145, 147 where incident to the lenses 151, 153. In an alternative embodiment, the lenses 151, 153 may be replaced with prisms that direct the laser beams 145, 147 without substantial focusing. Alternatively, decentered lenses may be provided that focus and deflect the laser beams 145, 147.

The lenses 151, 153 may be adapted so that a convergence point 165 of the beams 145, 147 may lie along an optical axis 171 of the object lens 113. The convergence point 165 may be at or near a focal point of the objective lens 113. Alternatively, in some applications the point 165 may not lie along the axis 171. For example, in applications involving the viewing of planar or substantially planar tissue features 108, the point may off the axis 171. In applications involving more three dimensional tissue features 108, the points 16 may be selected to lie along the optical axis 171.

A stereotactic wand may employ converging light beams in like fashion to the endoscope 100 described above. The light may be converged, for example, using lenses, prisms, or by proper formation of the light guides 141, 143.

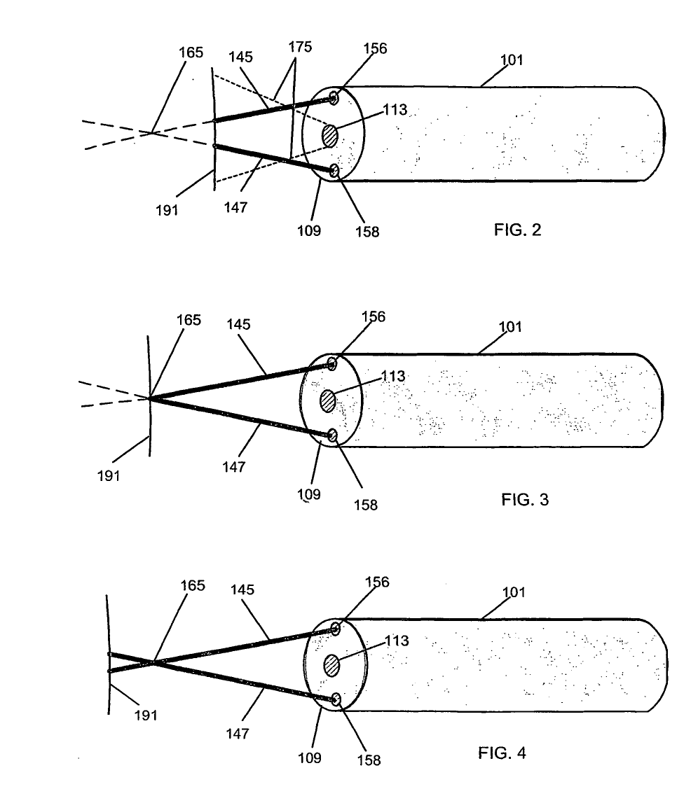

FIGS. 2-4 show the positioning of a light-assisted stereotactic device. In FIG. 2, a field of view 175 is collected by the objective lens 113. With endoscopes, the resulting image may appear, to some observers, substantially focused as viewed through an eyepiece or on a video monitor. However, the convergence point 165 of the beams 145, 147 is behind the feature 191. The beams 145, 147 do not appear to come to a point at the feature 191, indicating that the feature 191 is not situated at the predetermined distance from the end 109 of the device 101. Wands, while lacking an optical system to transfer the image to an observer, may comprise electronics to detect when the beams come to a point. Referring to FIG. 3, the device 101 has moved further away, in relation to the feature 191, than in FIG. 2. The feature 191 is situated at or near the predetermined distance from the end 109 of the device 101, and the beams 145, 147 appear to substantially come to a point 165. Referring to FIG. 4, the device 101 has moved even further away from the feature 191. The point 165 is now between the end 109 of the device 101 and the feature 191. Again, the beams 145, 147 no longer appear to intersect at the point 165, indicating that the end 109 of the device 101 is not substantially at the predetermined distance from the feature 191. Using the extent to which the beams are converged as an indication of the device 101 position removes some of the subjectivity associated with prior art stereotactic positioning techniques, particularly for endoscopes, which relied upon bringing the feature 191 into focus for an observer as an indication of the device's position.

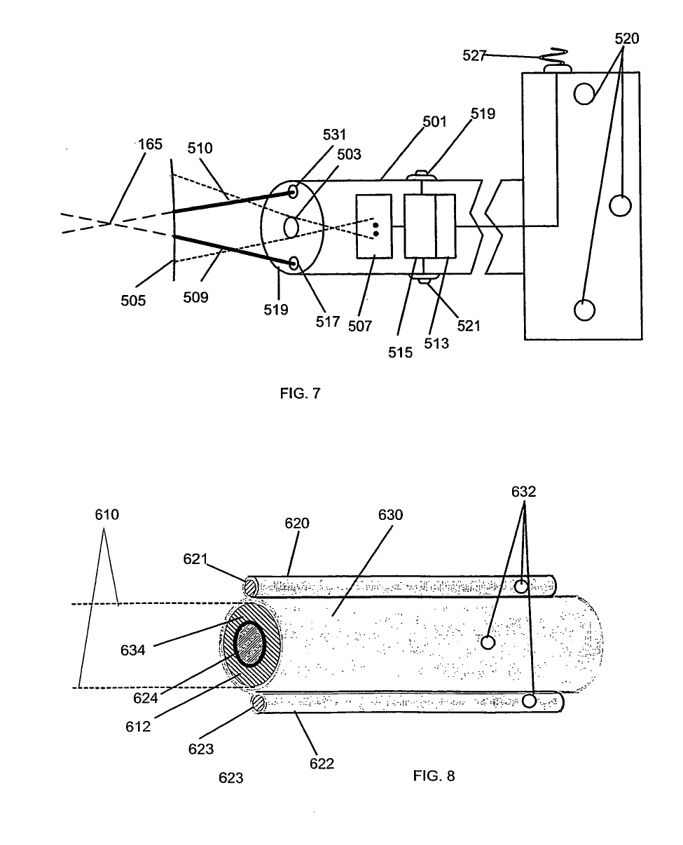

The device 101 may comprise electronics and logic to generate a signal when the beams 145, 147 are substantially converged at the point 165. The device 101 may further comprise electronics and logic to determine a direction to move the device 101 in order to more fully converge the beams 145, 147. Electronics and logic for this purpose are more fully described in conjunction with FIG. 7.

In one embodiment, the beams 145, 147 may have different visible color content. For example, the beam 145 may be blue and the beam 147 may be red. Thus, it may be possible to determine from the relationship of the beam points (blue on top or red on top) whether the device 101 is closer or further than the predetermined distance from the feature 191.

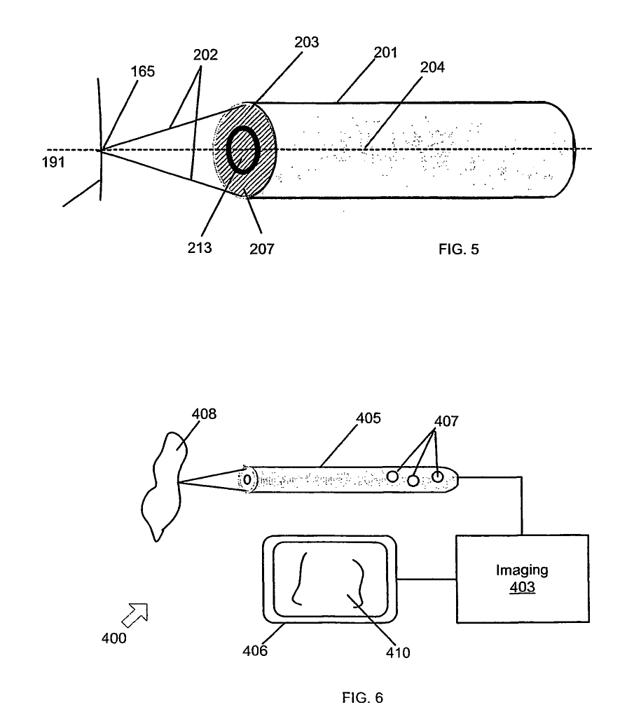

Referring to FIG. 5, in an alternative embodiment, a stereotactic surgical device 201 delivers light 202 to the feature 191 through an annulus 203. The light 202 may be produced, for example, using one or more lasers or light emitting diodes, or an incandescent source such as a quartz-halogen lamp, to name just a few of the possibilities. A light flux is directed by the annulus 203 in a converging fashion using, for example, a prismatic optical element such as one or more prisms, or an annular focusing lens. The annulus 203 may be configured to converge the light 202 to substantially a point, or to a ring or disk having an illumination area of a predetermined extent, at a predetermined distance from the distal end 207 of the device 201. With endoscopes and certain other types of surgical devices, the light 202 may also serve to illuminate the feature 191 as well as to ascertain when the distal end 207 is a predetermined distance from the feature 191.

Embodiments of the devices described herein may be employed in stereotactic systems. With reference to FIG. 6, a stereotactic system 400 includes an imaging system 403, a stereotactic wand 405, and a display 406. The display 406 may display an image 410 of the tissue feature 408. The imaging system 403 may be any imaging system suitable for body imaging such as systems using computerized tomographic (CT) methods, X-ray imaging, acoustic imaging, and magnetic resonance imaging (MRI). The stereotactic system 400 assists in the determination of the spatial locations of tissue features relative to (1) one another, and/or (2) the wand 405, and/or (3) an external reference point or points. The wand 405 may assist and/or improve upon such spatial determinations. For example, a position of a particular tissue feature having an MRI response similar to that of a surrounding or nearby tissues may be more accurately established by way of the wand 405.

The wand 405 may comprise adaptations in accordance with those described herein to assist in the positioning of the wand 405 at a predetermined distance from a tissue feature 408. The position of the wand 405 relative to the tissue feature 408 may be established without involving contact between the tissue 408 and the wand 405.

The wand 405 includes stereotactic markers 407 that are detectable by a stereotactic processor (not shown). The stereotactic processor determines the position of the wand 405 based on measurements of the absolute or relative positions of the stereotactic markers 407, in manners well known in the art. The stereotactic markers can be light emitters, light reflectors, or other positional references suitable for electromagnetic or other positioning systems. In one embodiment, light emitters are used and the positions of the light emitters are determined by imaging with a video camera or other light sensor.

Positioning the wand 405 at a predetermined distance from to the tissue feature 408 establishes the relative position of the tissue feature 408 with respect to the wand 405. Based on measurements of the stereotactic markers, the position of the wand 405 (and by extension, the feature 408 at a predetermined distance from the wand 405) may be determined relative to an external reference point, such as a stereotactic frame and the operating room. This process is often referred to as registration or co-registration of the wand with respect to the feature 408, external frame, and operating room. Thus, the wand 405 may be employed to establish positions of one or more tissue features, stereotactically. Surgical or other procedures may thus be planned and executed more precisely.

In operation, a physician or other operator may position the wand 405 to establish a predetermined spacing between the wand 405 and the feature 408, without contacting or deforming the tissue 408.

With reference to FIG. 7, a light-assisted automatic registration wand 501 includes an objective lens 503. A charge-coupled device (CCD) 507, or other suitable detector such as quadrant photodiodes, receives an image of a tissue feature 505 via the objective lens 503. The wand 501 may be configured to provide light beams 509, 510 from lenses 531, 517 which converge the beams 509, 510 at a point 165 a predetermined distance from an end 519 of the wand 501 (or from any reference location on the wand 505). The sensor 507 supplies a signal representing the image to a signal processor 515. Logic 513 is applied to the processor 515 to cause the processor 515 to determine whether the beams 509, 510 have converged to an extent which indicates that the wand 501 is at substantially the predetermined distance from the feature 505. When the processor 515 determines that the beams 509, 510 have sufficiently converged, a signal may be provided to an antennae 527 for transmission to a stereotactic system to effect automatic co-registration of the wand 501. Of course, alternate embodiments may operate in a tethered fashion (e.g. coupled by way of copper and/or optical signal conductors) to the stereotactic system, without employing an antennae 527.

The wand 501 may comprise a switch 519 which, when operated, may activate the processor 515 to determine when the beams 509, 510 are sufficiently converged, e.g. to initiate the process of automatic co-registration. Such activation may take place once an operator of the wand 501 determines, perhaps visually, that the beams are close to sufficiently converged. Thus, the operator may ‘rough position’ the wand 501 with respect to the feature 505, activate the processor 515, and then ‘fine position’ the wand 501 further until the signal is provided indicating that the beams 509, 510 are sufficiently converged. Another switch, 521, may be operated to manually produce the co-registration signal when the operator determines, in his or her own judgement, that the beams 509, 510 are sufficiently converged.

The logic 513 may also operate the processor 515 to determine a proper direction to move the wand 501 to effect automatic co-registration, the direction to move based upon the convergence or divergence of the beams 509, 510 as detected by the detector 507. For example, when moving toward the tissue feature 505, and the beams 509, 510 are diverging, the processor 515 may determine that the wand 501 direction should be reversed (e.g. the wand 501 is closer to the feature 505 than the predetermined distance). The processor 515 may provide a signal to indicate the proper wand direction, or to indicate that the wand direction should be reversed. This may be especially useful in very fine positioning applications, where it may be challenging to determine the convergence or divergence of the beams 509, 510 based upon a visual inspection alone.

Once a signal is provided, indicating that the wand 501 is positioned at the predetermined distance from the feature 505, the position of the feature 505 with respect to an external frame of reference is established based on stereotactic markers 520, effecting co-registration of the coordinates of the feature 505 with a stereotactic frame, the wand 501, and the operating room. In one application, the position of the feature 505 with respect to the wand 501 is used to confirm, establish, or modify the position of the feature 505 in a previously obtained image. The signal may be applied to activate a visual, tactile, or audible alarm (or a combination thereof) indicating the predetermined position has been obtained.

A similar set of electronics, controls, sensors, and logic may be employed in endoscopes and other surgical devices to effect automatic registration. In endoscopes, the light collected by the objective lens 503 may be split, with some of the light applied to the sensor 507 to effect automatic registration, while other of the light is transmitted via a fiber bundle or other mechanism to an observer.

With reference to FIG. 8, an embodiment 630 of a light-assisted stereotactic body 630 comprises light guides 620, 622 that provide light beams. Lenses 621, 623 are adapted to converge the beams at a predetermined position with respect to one of (1) a location on an endoscope 612, wand, cannula, or other device inserted within the body 630, and (2) a location on the stereotactic body 630. Alternatively, the light guides 620, 622 may be bent or otherwise adapted to converge the beams. The inserted endoscope 612, wand, or other device may comprise an objective lens 624 and an annulus 634 or other illumination source which provides a substantially non-converging light 610. Stereotactic markers 632 are provided on the stereotactic body 630 so that the enclosed endoscope 612, wand, cannula, or other device may be positioned using conventional stereotactic techniques. Thus, a conventional surgical device can be retrofitted to operate in accordance with the techniques described herein. Of course, the body 630 need not fully enclose the endoscope 612, wand, cannula, or other device, so long as the body 630 is securely fitted thereto.

The techniques described herein may be applied with wands, endoscopes, and other surgical devices including flexible, semirigid, and rigid body types. Flexible and semi-rigid devices can be provided with registration systems that permit determination of the position of the distal ends of the devices with respect to the stereotactic markers. The registration systems can be implemented as mechanical systems or can be based on electromagnetic, optical, or other sensor systems. With such registration systems, the position of the intersection point or convergence point of one or more light fluxes with respect to the stereotactic markers can be established.

In view of the many possible embodiments to which the principles of the present invention may be applied, it should be recognized that the detailed embodiments are illustrative only and should not be taken as limiting in scope. Rather, the present invention encompasses all such embodiments as may come within the scope and spirit of the following claims and equivalents thereto.

What is claimed is:

1. A stereotactic surgical device comprising at least one lens to converge light to a predetermined extent at a predetermined distance from a predetermined location on the device.

2. The device of claim 1 further comprising:
a plurality of light guides to provide a plurality of beams; and
the at least one lens to converge the plurality of beams to the predetermined extent at the predetermined distance.

3. The device of claim 2 wherein the beams comprise different visible color content.

4. The device of claim 1 wherein the at least one lens comprises:
an annulus to converge the light to the predetermined extent at the predetermined distance.

5. The device of claim 2 wherein the beams comprise laser light and the predetermined extent comprises substantially a point.

6. The device of claim 5 further comprising:
at least one laser diode to provide the laser light.

7. The device of claim 1 further comprising:
a light detector; and
a processor to receive image information from the light detector; and
logic which, when applied to the processor, causes the processor to determine when the light has converged to the predetermined extent and to provide a signal thereupon.

8. The device of claim 7 wherein the signal is applied to provide automatic co-registration of the device with respect to at least one of a feature to image, an external frame, and an operating room.

9. The device of claim 7 further comprising:
a control which, when operated, enables the processor to begin determining when the light has sufficiently converged.

10. The device of claim 7 further comprising:
a control which, when operated, causes the signal to be provided in bypass of the processor.

11. The device of claim 7 further comprising:
logic to determine a proper direction to move the device for co-registration based upon convergence and divergence of the light.

12. An apparatus comprising:
a stereotactic body comprising
an opening to receive one of an endoscope, a wand, and a cannula; and
at least one lens to converge light to a predetermined extent at a predetermined distance from a predetermined location on one of the endoscope and the stereotactic body.

13. The apparatus of claim 12, the at least one lens comprising:
a plurality of light guides to provide a plurality of beams; and
the at least one lens to converge the plurality of beams to the predetermined extent at the predetermined distance.

14. The apparatus of claim 13 wherein the beams comprise different visible color content.

15. The apparatus of claim 12 wherein the at least one lens comprises:
an annulus to converge the light to the predetermined extent at the predetermined distance.

16. The apparatus of claim 13 wherein the beams comprise laser light and the predetermined extent comprises substantially a point.

17. The apparatus of claim 17 further comprising:
at least one laser diode to provide the laser light.

18. The apparatus of claim 12 further comprising:
stereotactic markers.

19. A stereotactic system, comprising:
an imaging system configured to form an image of a tissue feature located at a predetermined distance from a location on a stereotactic surgical device; and
a focus system that provides one or more light fluxes that converge at an intersection at the predetermined distance.

20. The stereotactic system of claim 19, further comprising an alarm configured to indicate that the tissue feature is positioned at the predetermined distance.

21. The stereotactic system of claim 19, further comprising:
a light detector; and
a processor to receive image information from the light detector; and
logic which, when applied to the processor, causes the processor to determine when the light has converged at the intersection and to provide a signal thereupon to effect co-registration of the device.

22. A stereotactic surgical device comprising at least two light guides adapted to converge light to a predetermined extent at a predetermined distance from a predetermined location on the surgical device.

23. The surgical device of claim 22 wherein the light comprises:
two beams of different visible color content.

24. The surgical device of claim 22 wherein the light guides comprise:
optical fibers bent to converge the light to the predetermined extent at the predetermined distance.

25. The surgical device of claim 23 wherein the beams comprise laser light and the predetermined extent comprises substantially a point.

26. The surgical device of claim 25 further comprising:
at least one laser diode to provide the beams.

27. The surgical device of claim 22 further comprising:
a light detector; and
a processor to receive image information from the light detector; and
logic which, when applied to the processor, causes the processor to determine when the light has converged to the predetermined extent and to provide a signal thereupon.

28. The surgical device of claim 27 wherein the signal is applied to provide automatic co-registration of the device with respect to at least one of a feature to image, an external frame, and an operating room.

29. The surgical device of claim 27 further comprising:
a control which, when operated, enables the processor to begin determining when the light has sufficiently converged.

30. The surgical device of claim 27 further comprising:
a control which, when operated, causes the signal to be provided in bypass of the processor.

31. The surgical device of claim 27 further comprising:
logic to determine a proper direction to move the device for co-registration based upon convergence and divergence of the light.
